(19) United States
(12) Patent Application Publication
(10) Pub. No.: US 2021/0287800 A1
(43) Pub. Date: Sep. 16, 2021

(54) AI SUPPORTED PERSONALIZED, NATURAL LANGUAGE-BASED PATIENT INTERFACE FOR MEDICAL-BOT

(71) Applicant: MEDIUS HEALTH, North Sydney (AU)

(72) Inventors: Shameek GHOSH, North Sydney (AU); Budhaditya SAHA, North Sydney (AU)

(73) Assignee: MEDIUS HEALTH, North Sydney (AU)

(21) Appl. No.: 17/196,936

(22) Filed: Mar. 9, 2021

Related U.S. Application Data
(60) Provisional application No. 62/987,833, filed on Mar. 10, 2020.

(51) Int. Cl. G16H 50/20; G16H 10/20; G16H 70/20; G16H 10/60; G16H 50/30; G16H 50/70; G06N 5/02; G06F 16/2452; G06F 40/205

(52) U.S. Cl. CPC G16H 50/20; G16H 10/20; G16H 70/20; G16H 10/60; G06F 40/205; G16H 50/70; G06N 5/02; G06F 16/24522; G16H 50/30

(57) ABSTRACT

A personal medical-bot with a natural language translator implemented on a personal communication device. The medical-bot interacts in natural language with a user respondent/patient who presents a medical problem. The medical-bot includes a natural language translator with an artificial intelligence (AI) module that accepts the natural language inputs, and identifies medically relevant terminologies and their associations. These are fed to the AI for processing generate clinical-based queries to be answered by the patient. The responses are used by the medical-bot to extract medically relevant data for establishing a medical history and enabling a medical diagnosis for the patient. The medical-bot is able to simulate the sequential queries of a doctor or nurse practitioner to arrive at a diagnosis and an immediate treatment plan, which determines the triage to be followed by the patient. A health score for the patient is also determined.

### PRIORITY

The present application claims priority to U.S. Provisional Application No. 62/987,833, entitled “AI Supported Personalized, Natural Language-Based Patient Interface for Medical-Bot,” filed Mar. 10, 2020, the entirety of which is hereby incorporated by reference.

### BACKGROUND

The need for medical professionals is fast out-pacing the availability. In many parts of the world, especially in poorer countries, there are fewer than one doctor per 10s of thousands of individuals. In certain other places, vast distances prevent medical practitioners from attending to patients in a timely fashion. Hence, there is a great need for an AI based capability that can be used to diagnose patient's current illness and future risk of diseases.

### SUMMARY

Embodiments of the invention are directed to an AI based capability that can be used to diagnose patient's current illness and future risk of diseases. The system is able to engage a patient in an interactive fashion by generating relevant queries based on the patient's prior input or answers, similar to the interaction in a doctor's office to extract information, and to arrive at a diagnosis of the patient's problem and also report an overall health score enabling risk assessment of the patient. This interactive system is of great use, particularly in remote locations and in locations where immediate access to a medical practitioner is limited. Further, it encourages better awareness of health by gamification.

More particularly, embodiments of the invention are directed to the use of a personal medical-bot with a natural language translator is implemented on a personal communication device to interact with a user who wants to present a medical problem as a patient or identify a medical risk for himself as a respondent.

Embodiments of the invention are directed to the use of a natural language translator coupled to the artificial intelligence (AI) module forming the medical-bot to accept natural language inputs, identify medically relevant terminologies and associations between those detected terms.

Embodiments of the invention are directed to the capability to feed medically relevant information collected in the form of a patient's inputs to the AI for processing to retrieve information and generate clinically relevant queries for answer by the patient using inputs from a vector knowledge graph.

Embodiments of the invention are directed to the use of the query responses by the medical-bot, with the AI capability, to extract medically relevant data within the vector knowledge graph for establishing a medical history, a medical diagnosis of the patient's problem. The medical-bot may be enabled to simulate the sequential queries of a general practitioner or a nurse practitioner to arrive at a medical history, that points to a diagnosis.

Embodiments of the invention are directed to the use of the interactive query-responses by the medical-bot, with the AI capability, is used to extract medically relevant data on the current illness of the patient. It is also simultaneously able to generate a health score that quantifies the future health risk of the patient. This health score generation is accomplished by interactively extracting personal features of the patient such as, height, weight, obesity index and also other medically relevant behavioral characteristics of the patient relating to his habits of smoking, drinking, food habits, frequency of exercise etc. that impact the overall health of the patient or respondent.

Embodiments of the invention are directed to systems and processes that provide an immediate treatment/action plan to be acted on by the patient. The action plan determines the triage to be followed by the patient.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 is a block diagram of an intelligent medical system in accordance with one embodiment of the invention.

FIG. 2 is an exemplary flow chart of a diagnostic and response capability in accordance with one embodiment of the invention

FIG. 3 is a block diagram of an exemplary flow of operation of the system leading to diagnosis of a patient's problem in accordance with one embodiment of the invention.

FIG. 4 is an exemplary set of queries generated based on a user input for initial diagnosis and for confirming the most reasonable diagnosis in accordance with one embodiment of the invention.

FIG. 5 is an example of tracking software used to track interactions including queries and responses from a patient user leading to a diagnosis and treatment plan that is provided to the patient user in accordance with one embodiment of the invention.

FIG. 6 is a simplified example illustrating use of a knowledge graph to arrive at a possible final diagnosis from an initial symptom input from a user patient in accordance with one embodiment of the invention.

FIG. 7 is a schematic illustration of an exemplary personal communication device for implementing the intelligent medical system in accordance with one embodiment of the invention.

### DETAILED DESCRIPTION

Embodiments will be described below in more detail with reference to the accompanying drawings. The following detailed descriptions are provided to assist the reader in gaining a comprehensive understanding of the methods, apparatuses, and/or systems described herein and equivalent modifications thereof. Accordingly, various changes, modifications, and equivalents of the methods, apparatuses, and/or systems described herein will be apparent to those of ordinary skill in the art. Moreover, descriptions of well-known functions and constructions may be omitted for increased clarity and conciseness.

The terms used in the description are intended to describe embodiments only, and shall by no means be restrictive. Unless clearly used otherwise, expressions in a singular from include a meaning of a plural form. In the present description, an expression such as “comprising” or “including” is intended to designate a characteristic, a number, a step, an operation, an element, a part or combinations thereof, and shall not be construed to preclude any presence or possibility of one or more other characteristics, numbers, steps, operations, elements, parts or combinations thereof.

Embodiments of the invention relate to a medical-bot that is an AI based virtual medical assistant for identifying and diagnosing a patient's condition and the patient's future health risk based on interactive queries generated by a medical bot and answers from the patient. The medical bot is enabled, based on the diagnosis to provide a triage recommendation to the patient. The medical bot based on the interactive query-response is further able to identify the patient's characteristics including behavioral characteristics that can impact health of the patient in future, such as lack of exercise, smoking, drinking etc. and generate a health score for the patient that is indicative of the health risk the patient has.

The medical-bot is enabled with artificial intelligence (AI) software with sufficient processing capability in modules of the medical bot requiring an intelligence and decision making capability. The medical-bot is able to interact in natural language with the patient user who wants to present a medical problem. The AI is intelligent software instantiated as part of the medical-bot. The AI intelligence is provided by the various modules of the bot discussed in further below that practice the process and perform the intelligent decision making. The artificial intelligence is executed by the processing systems with software that is able to perform tasks that normally require human intelligence, such as visual perception, speech recognition, decision-making, and translation between languages (e.g., natural language to machine language and vice versa). The personal medical-bot typically includes a natural language translator.

The personal medical bot may be implemented on a personal communication device, such as a laptop or mobile phone, of a user patient. The medical-bot interacts in natural language with a patient user who wants to present a medical problem, find a diagnosis, and receive an action plan. The natural language translator in the AI module on the medical-bot accepts the natural language inputs from the patient user and identifies the medically relevant terminologies conveyed in the natural language as also their association within the natural language inputs.

These are provided to the AI for processing to retrieve information and generate intelligent clinical based queries. Answers to these queries by the user patient are used to clearly define the user patient's medical history and ailment. More specifically, the responses are used by the medical-bot to extract medically relevant data for establishing the medical history of the patient and enabling a medical diagnosis of the patient's problem.

The medical-bot has a policy engine enabled with the AI that is programmed to be able to use a relevant section of a clinical knowledge graph to simulate the sequential queries of a doctor who is a general practitioner or a nurse practitioner questioning the patient. The answers enable the medical-bot to process the answers and move through the clinical knowledge graph to arrive at a diagnosis, future disease risks, a health score, and an immediate treatment plan to be acted on by the patient user. The action plan determines the triage to be followed by the patient.

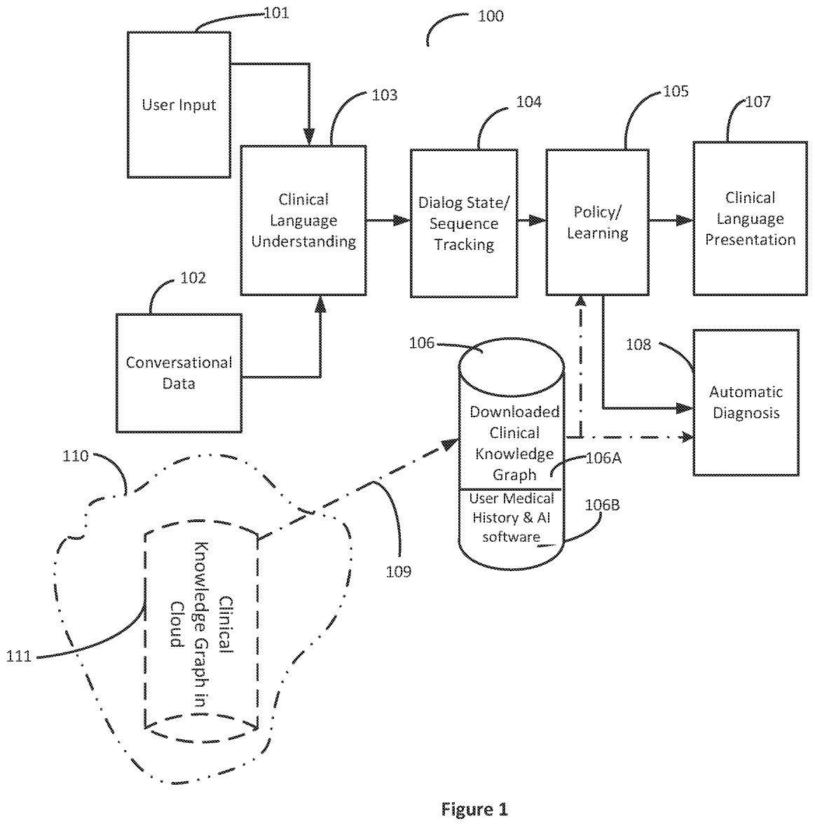

FIG. 1 is an exemplary block diagram representation of an intelligent personal medical-bot system (100) instantiated on the user's communication device or on a stand-alone communication enabled device, having sufficient computing capability, typically the system (100) is instantiated on a mobile communication device (MCD) of the user, such as a cell phone or tablet. The personal medical-bot is enabled to emulate a medical assistant/general practitioner to receive patient inputs and arrive at a diagnosis and plan of action.

The intelligent medical-bot system (100) has built-in artificial intelligence (AI) and uses the clinical knowledge graph (KWG) capability and is capable of accepting natural language inputs, generate queries for clarification and arrive at a diagnosis, future disease risks, health score and treatment plan for the user-patient of the mobile communication device.

As shown in FIG. 1, the intelligent personal medical-bot (IPMB) system (100) includes a number of components to accomplish its tasks as shown in Table 1 below:

TABLE 1

| Components | Description | Ref. # |
|---|---|---|
| User input module | Module to receive input from the patient user | 101 |
| Conversational module | Collects the conversational input from the patient user for | 102 |
| | use in analysis | |
| Clinical Language | Processes the user input data into machine readable | 103 |
| understanding (CLU) | format having a dictionary like structure having intent and | |
| module | slots as keys (shown in Table 2) | |
| Dialog state/sequence | Tracks the sequence of the dialog between user and the | 104 |
| tracking Module | bot, tracking the query answers, checks for coherency | |
| | before inputting to the AI | |
| Policy/learning Module | Includes AI capability that takes the inputs and traverses | 105 |
| | the stored knowledge graph based on the established | |
| | policies of the AI | |
| Data Store | A data store for downloading and storing an applicable | 106 |
| | knowledge graph section for use during an interaction | |
| | with the patient and software of the AI associated with | |
| | the modules of the Medical Bot (106A). The data | |
| | store also keeps the historic data record of diagnosis | |
| | arrived at for the user patient (106B). | |
| Clinical Language | Presents the queries generated by the AI to the user in a | 107 |
| presentation module | natural language for response | |
| Automatic diagnosis and | Generates and automatic diagnosis, a disease prediction, | 108 |
| risk prediction module | health score and a triage for action to be presented to the | |
| | user as an end product | |

The IPMB 100 includes a user input module (101) that is a natural language input module for receiving natural language input from the patient user.

The received inputs are then fed to the clinical language understanding module (103). The clinical language understanding module (103) is coupled to a conversational data module (102) that collects and stores medical conversational language terminology that is relevant and helpful in understanding of the natural language inputs form patient users received via the input module (101). The conversational data collected and stored in the conversational data module (102) is used to decipher the medical related terminology used in the vocabulary of the local area, as the colloquial natural language inputs for medical terms can differ widely over regions of the same country, let alone across country borders by the clinical language understanding module (103). The availability of inter-relatable medical terminology, from the conversational data module (102) helps the clinical language understanding module (103) to recognize, understand, standardize and convert the medically relevant natural language input data from the patient user. This clinical language understanding module (103) hence converts the nonstandard conversational input from the patient user into a standardized digital data format that is machine readable and usable for data processing.

In one embodiment, the conversion from natural language to digital data format by the clinical language understanding module (103) uses a slot filling technique that enables the data in the natural language input to be separated into key components. In one particular embodiment, the key components are separated into one of three categories such as an IOB or D (In/Out/Begin or Descriptor representation): (“In” or intent symptoms, I), (symptom descriptors D or the beginning of symptoms B), and (language structure or “Out,” 0). Table 2, below, illustrates an example of the slot filling process division into the key components for a natural language input from a user stating “I have stomach pain with continuous fever”. The slot filling process is used to convert the natural language input from user into digital data and also to convert digital data to be output to natural language output to the user.

TABLE 2

| | | | | | | | |
|---|---|---|---|---|---|---|---|
| Sentence | I | Have | Belly | Pain | with | continuous | fever |
| Slots | O | O | D-Symptom | I-Symptom | O | D-Symptom | D-Symptom |

| | |
|---|---|
| Intent | I-Symptom |
| Descriptor | B or D-Symptom |
| Language | O-structure |

A further description of slot filling will now be provided. The semantic parsing of a sentence in natural language includes three tasks: domain detection, intent determination, and slot filling. Particularly, slot filling is the task of assigning a domain relevant entity tag or type (also known as a slot) to a word (or token) in a sentence. In machine learning, slot filling is typically treated as a sequence classification problem in which each word in sentence is assigned a semantic label. For example, as shown in Table 2, the sentence is “I have belly pain with continuous fever”. Here, the phrases “belly pain” and “continuous fever” are symptoms as described in the domain of medicine. Accordingly, a symptom can be represented by the domain tag—“symp”. Further, (an IOB or IOD (In/Out/Begin or Descriptor representation) tagging method is used where B-symp denotes the beginning of a symptom tag, I-symp denotes being inside the symptom tag, and O denotes anything outside of these types. Thus in table 2, “belly” is labelled as “B or D-symp”, “pain” is labelled as “I-symp”, and non-symptom words are labelled as “O”. Thus, slot-filling using clinical tags is an important problem that is solved using a Clinical Language Understanding (CLU) module.

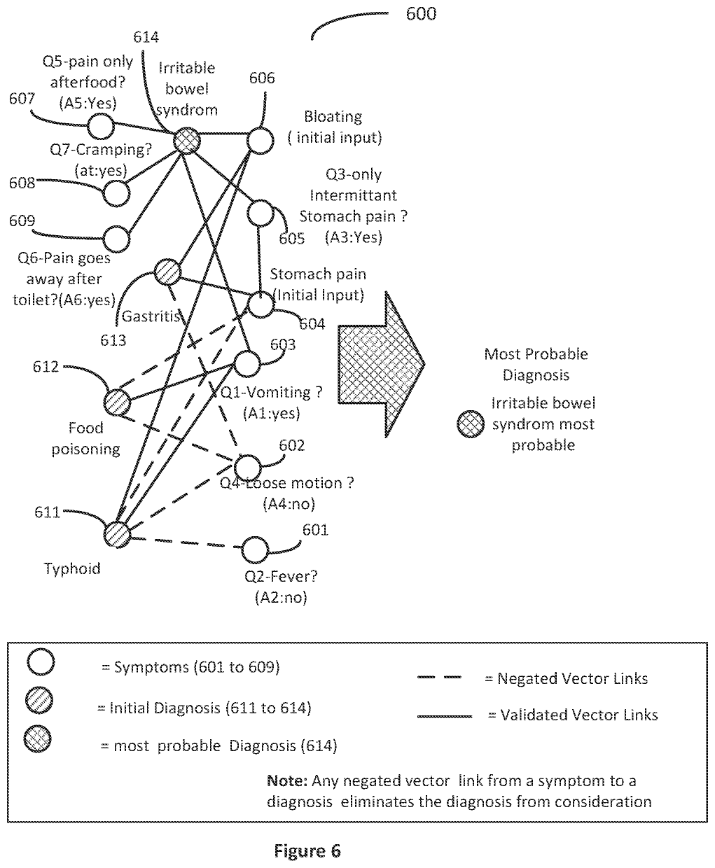

The converted digital data is sent to a dialog state and sequence tracking module (104) that is enabled to track the interaction of the personal medical-bot with the patient during their interaction. This tracking is used to ensure that the queries provided to the patient and the answers are co-relatable and coherent as the interactions progress. The dialog state and sequence tracking module (104) checks the inputs for coherence and passes them on to an AI based policy learning module (105) that is coupled to a clinical knowledge graph (106A). Additional details about the knowledge graph (106A) are described in further detail below and with reference to FIGS. 6 and 6A

It should also be understood that the full clinical knowledge graph (111) is a very large collection of clinical knowledge in digitized form and is typically stored in the cloud (110) for access by the user and the medical bot. On receipt and analysis of the initial symptoms the policy/learning module (105) of the medical bot is enabled to connect via a wireless communication link (109) from the user device to the full clinical knowledge graph (111) in the cloud (110) to request and download a relevant section of the clinical knowledge graph (106A) having relevance to the symptoms and diagnosis to the data store (106) on the user device. This is done to speed up the diagnostic process for the user patient. Further downloads of sections of the clinical knowledge graph data can be requested at a later time if the policy module finds it necessary. Though the preferred method is to download the relevant section of the clinical knowledge graph into the data sore of the user device it should not be considered mandatory as it is possible to access the clinical knowledge graph remotely in the cloud and use the capabilities if needed. The data store (106) on the user device also has a partition that stores the user's medical history and the AI software (106B).

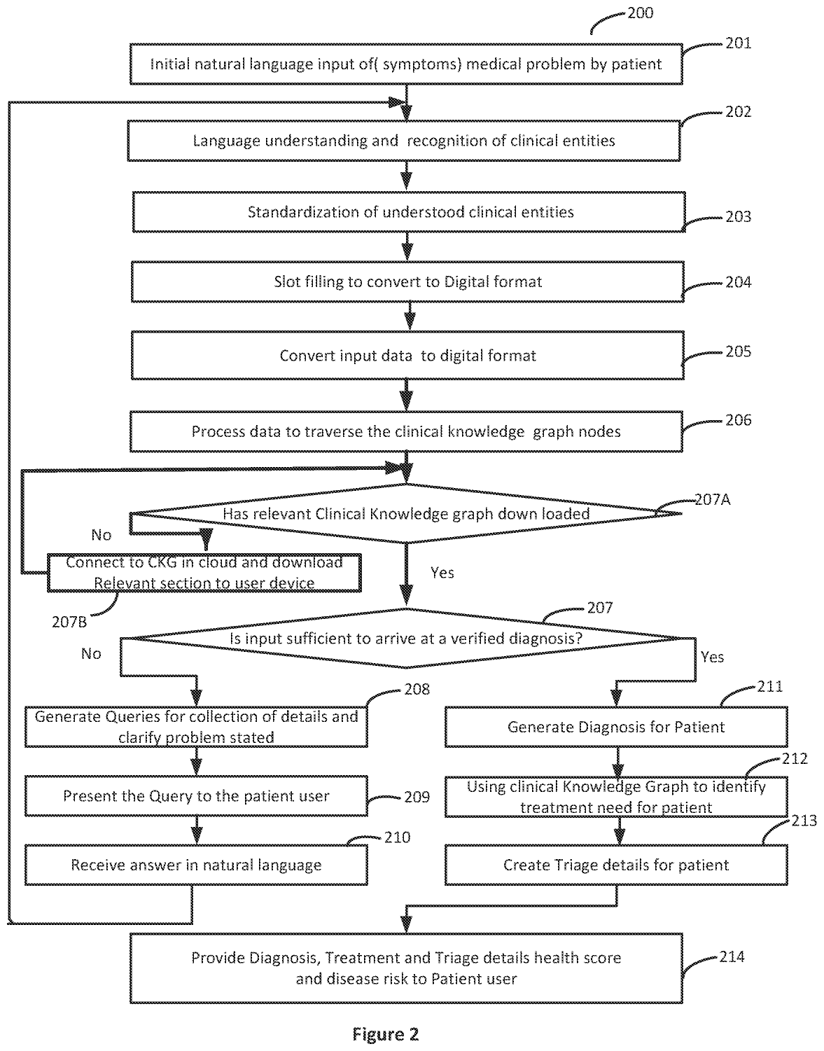

FIG. 2 is an exemplary flow chart (200) of the sequence of operations of the IPMB system (100). It will be appreciated that the sequence of operations may vary from that shown in FIG. 2 and that there may be additional or fewer operations than that shown in FIG. 2.

As shown in FIG. 2, the natural language input is received by the IPMB system (201). The inputs are typically the initial description of the symptoms of the medical problem faced by the patient (may not be all existing symptoms, in which case queries are used to clarify/extract the symptoms).

The process continues with the received input being processed to understand the clinical terminology and usage within the natural language input (202). The inputs are then converted into a standardized form (203). This standardized form is then used for slot filling (204) to identify the digitizable elements in the standardized input form, as discussed above with reference to, for example, Table 2.

The digitizable elements identified by the slot filling process are converted into a digital data format (205) usable by the digital modules of the IPMB (100). The digital modules then use the provided digital data as inputs to initiate traversal of the vectors linking the nodes of the digital graph (206).

Since the full clinical knowledge graph consists of massive compilation of available medical knowledge, it is stored in the cloud (110) in distributed storage (111) and is made accessible over wireless communications (109) to the user's device in order to speed up the process of diagnosis, the initial symptoms based digital data from the input by the patient user is provided to the clinical knowledge graph (111) in the cloud over the communication channel (109). The clinical knowledge graph (111) in the cloud (110) extracts relevant sections of the graph and downloads it into the local data store 106 on the user device as clinical knowledge graph 106A. Unless additional information from the full knowledge graph becomes necessary, the downloaded and locally stored relevant section of the clinical knowledge graph (106A) is used to determine a diagnosis for the patient user.

In order to ensure that the relevant section of the knowledge graph is downloaded and available on the data store of 106 of the user patient a check of the availability is done (207A) once the initial symptoms data from the patient is ready to be input into the clinical knowledge graph (206). If the relevant portion of the clinical knowledge graph (106A) is not yet down loaded and available on the data store 106, the digital form of the input at (206) is transferred to the full clinical knowledge graph (111) in the cloud (110) over the wireless communication link (109) of the user device to enable the clinical knowledge graph (111) in the cloud (110) to identify and selectively download (207B) the relevant portions of the clinical knowledge graph (106A) into the data store 106 of the patient user. This downloaded section of the clinical knowledge graph (106A) is for use in the diagnostic process by the IPMB (100) and it is locally accessible from the data store 106. Once the relevant portions of the clinical knowledge graph (106A) is in the data store 106 the process continues using the locally available clinical knowledge graph (106A) as bellow.

The input digital data is checked to see if it will enable convergence within the clinical knowledge graph to a diagnostic node; that is, the medical bot determines if the available data is sufficient to arrive at a diagnosis of the patient user's problem (207).

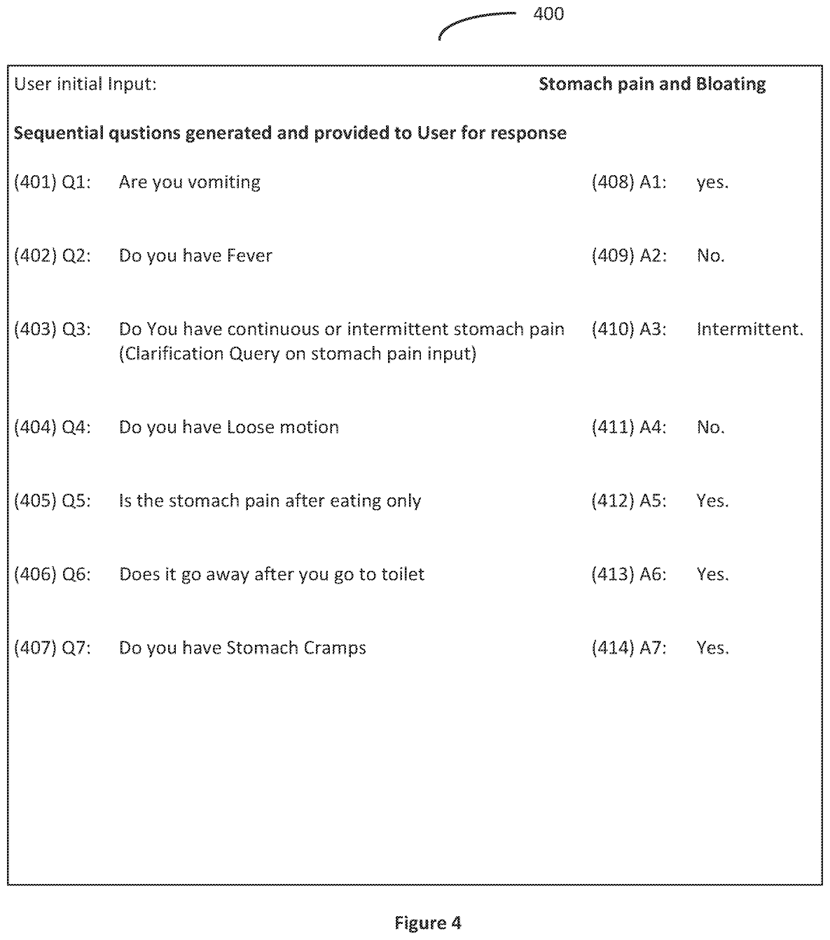

If the data is found to be insufficient to arrive at a diagnosis, the digital modules with the associated AI identify the plurality of paths/links that form the interconnecting and linking vectors of the clinical knowledge graph that limit a convergence and develop queries to be presented to the patient user in a sequential fashion. These queries (401 to 407 in FIG. 4) that can elicit additional inputs as answers (408 to 414 in FIG. 4) from the patient user are used to clarify the symptoms (208). The queries are designed to eliminate alternate paths from nodes and hence provide a convergent path to an initial diagnosis. The developed queries are presented to the patient, preferably in natural language form for answering (209). Answers from the patient user is received (210) and it is fed back for processing through the steps (202) to (207) until the cumulative data input into the clinical knowledge graph is sufficient to arrive at a diagnosis at (207). Once the data availability for convergence to a diagnosis to be achieved, it is used to generate a diagnosis. This diagnosis is checked to confirm that it is the most probable diagnosis by use of queries that check linking from other available symptoms.

If the diagnosis is not fully verified additional queries are generated to verify that the diagnosis is correct by again getting responses from the patient user and following the flow path from (208) to (202) and to (207) to complete the verification of the diagnosis.

The verified diagnosis from the clinical knowledge graph is used to answers any questions of the patient user and provide the diagnosis for the patient's illness (211). The diagnosis is further used within the clinical knowledge graph to search for a treatment plan for the diagnosed problem (212). A triage is developed and output to the patient (213). The diagnosis, the treatment proposed, and the triage are all provided to the patient user for follow through (214).

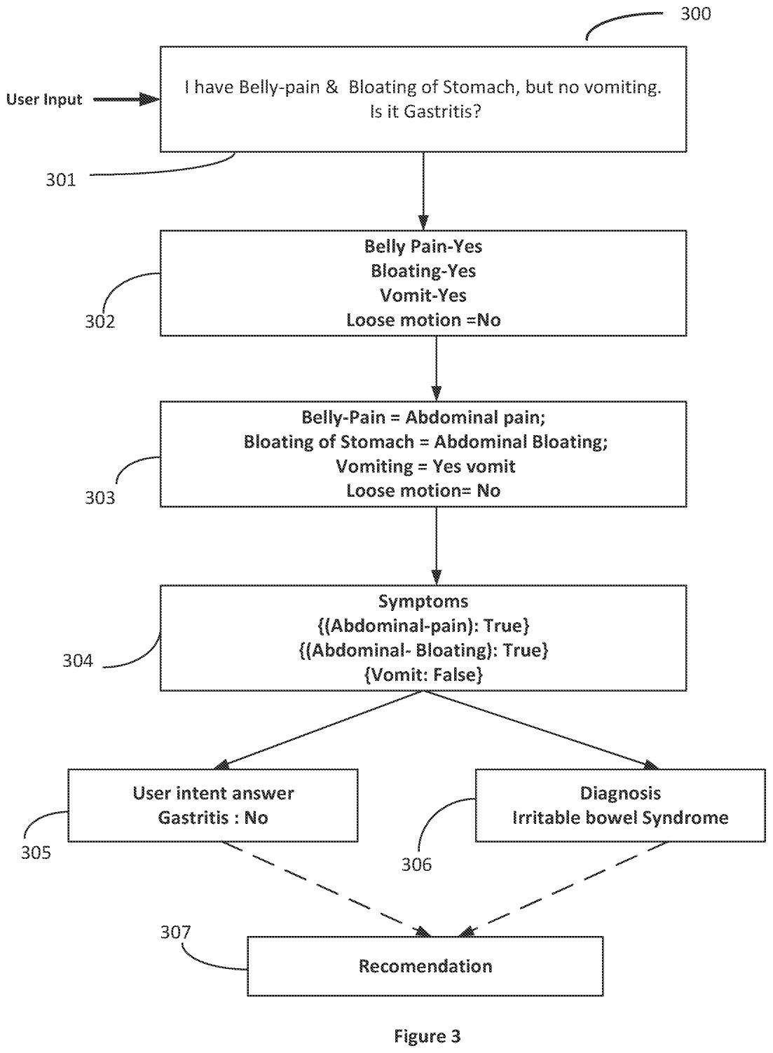

FIG. 3 illustrates an exemplary diagnostic flow from input of an initial description of the problem (e.g., “I have belly pain and bloating but no vomiting; Is it gastritis”) to a diagnosis and treatment plan using the IPMB 100.

As shown in FIG. 3, the process begins with a possible input into the user input module 101 the question from the patient in natural language with his identified symptoms of stomach pain and bloating, whether it is some disease he recognizes such as “gastritis” (301).

This input data is used to extract the clinically relevant entities from the natural language form such as “belly pain”, “bloating,” and “vomiting” (302) in the conversational module 103.

The clinically relevant information is also standardized at this time within the module 103 to enable conversion into digital format, “belly pain” being standardized as “stomach pain”, “bloating” as “stomach bloating or extension” and “no vomiting” as “no vomiting” (303).

The standardized sentences enable slot filling and clear identification of symptoms presented (404) which are then converted to digital format within the module 102.

The digital format when input into the relevant section of the clinical knowledge graph from the storage 106 by the policy module 105 to generate queries that are provided to the user via the clinical language presentation module 107 to receive responses and enable convergence to a result that is the diagnosis.

As can be understood the dialog state and sequencing module 104 keeps track of the interaction between the bot and the user to enable an effective and cohesive understanding of the interactions. Once a diagnosis is arrived at that is fed to the automatic diagnosis module 108 for further data extraction from the knowledge graph to establish the triage and the health score. This may or may not require additional queries and responses between the bot and the user.

In this exemplary case, the result indicate that the diagnosis does not support the patient user's assumption of gastritis (305) but point to “irritable bowel syndrome” (306). A treatment plan is also extracted from the clinical knowledge graph for the diagnosis reached and a triage recommendation is provided to the patient. (307).

FIG. 4 is an example (400) of the queries (Q1 to Q7, 401-407) sequentially generated for collecting answers (A1 to A7, 408-414) form the patient user in order to arrive at a diagnosis and to verify that it is the most probable diagnosis. These queries are generated to validate or negate known vector linkages within the clinical knowledge graph to enable convergence to a diagnosis. An example of the convergence process using queries and responses including the development of queries (Q1 to Q7, 401-407) is illustrated and described with reference to FIG. 6. The queries (Q1 to Q7, 401-407) are generated sequentially based on previous input and the vector linkages that have not been verified or eliminated in the clinical knowledge graph. This relationship is also illustrated with reference to FIG. 6. The queries (Q1 to Q7, 401-407) are generated sequentially based on previous input and the vector linkages that have not been verified or eliminated from consideration for arriving at a diagnosis in the clinical knowledge graph. This relationship is also illustrated in FIG. 6 showing the interlinked structure of the clinical knowledge graph (600) and of its operation to arrive at a most probable diagnosis.

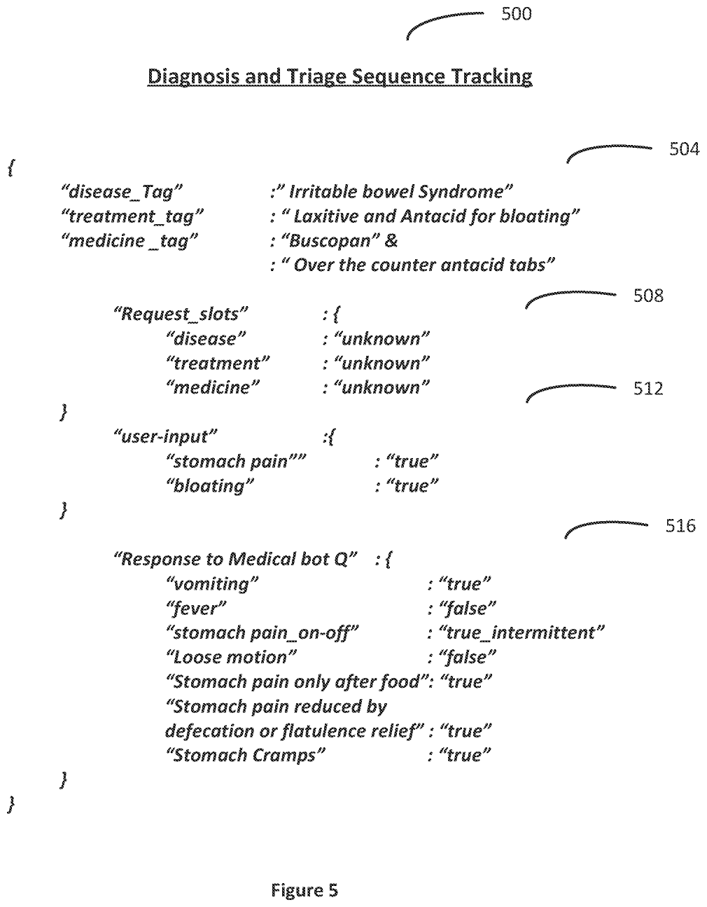

FIG. 5 shows a simplified sample (500) of a software algorithm used to track the interaction between the IPMB 100 and the user by the learning/tracking module (104). The algorithm illustrated in FIG. 5 shows the software used track the interaction between a patient user and the IPMB 100. The diagnosis and treatment plan 504 based on the original user inputs 512 and the medical bot user inputs 516 (e.g., response-A1 to response-A7 (408-414) in response to query-Q1 to query-Q7 (401-407) from FIG. 4) from a patient user. In the example shown in FIG. 5, the diagnosis and treatment plan 504 (e.g., the disease_tag is “Irritable Bowel Syndrome,” the treatment_tag is “Laxative and Antacid for bloating,” and the medicine_tag is “Buscopan” and “Over the counter antacid tabs” for the user information provided in the example). The disease_tag, treatment_tag and medicine_tag are provided in response to the “request_slots” 508 based on the “user-input” and the “response to Medical bot Q (questions)” as shown in FIG. 5. The diagnosis and treatment plan 504 is provided to the patient user in accordance with one embodiment of the invention. The collected information is also extracted at the end of the process and saved as part of the user medical history data in the data store 106b on the user device.

The interactions include at least the output of the diagnosis, and the treatment recommendation arrived at as provided to the user as well as queries to the user and responses to the queries from the user. Once the most probable diagnosis is reached the clinical knowledge graph is used to identify the need for treatment and generate the treatment/triage for the patient user is delivered to the user, the data collected and tracked by the program is extracted and saved to the user medical history (106B) in the local data store 106 of the user device for future use.

At this point the data extracted from the clinical knowledge graph is input into the slot fill to convert to natural language by the policy/learning module (105) to be presented to the user by the clinical language presentation module (107). In addition to the diagnosis and treatment arrived at a health score and disease risk value are also extracted from the clinical knowledge graph and converted to natural language format to be presented to the patient.

As discussed above, the downloaded relevant section of the clinical knowledge graph (106A) downloaded into the device data store 106 of the user device from the clinical knowledge graph (106A) for the use by the application and is used to generate queries for the user in response to each of his answers in a sequential manner to eliminate alternate diagnosis that may be available and arrive at a most probable diagnosis for the patient's condition. This diagnosis is then used to generate the treatment plan and health score for the patient. The clinical knowledge graph (111 and 106A) are built up in a vector graph form, of clinical symptoms, disease understanding, and treatment options collected from all available sources of medical knowledge. The graph is interlinked based on relationships by vector connectors or links that allow for validation or elimination of these linkages using answers to queries.

The AI based policy learning module (105) is able to take the symptom inputs received from the patient user and traverse the clinical knowledge graph (106A) to intelligently generate queries that help it to traverse to nodes of the graph using high probability paths to a disease identification node. The AI based policy learning module (105) is able to generate queries for the user for answers, that help it to eliminate paths to different nodes within the clinical knowledge graph when there are multiple nodes that can be reached with similar probabilities using available inputs from the patient user.

These queries and final diagnosis and findings are presented to the user via an output module designated clinical language presentation module (107). The queries are typically presented to the user in a natural language form, where possible.

Using the question-answer interaction between the medical-bot and the patient user, the AI-based policy learning module (105) is able to traverse the clinical knowledge graph (106A) to reach a disease diagnosis.

This data is output to an automatic diagnosis and risk prediction module (108). The automatic diagnosis and risk prediction module (108) is further enabled to access the knowledge graph (106A) to extract the treatment and triage options for a disease diagnosed for the patient. This data is extracted by the automatic diagnosis module (108) and presented to the patient user as a treatment plan and triage option for follow up by the patient user (in addition to a disease risks and health score based on the current diagnosis combined with the stored historic medical data of the patient).

A clinical knowledge graph represents a collection of interlinked descriptions of entities—objects, events or concepts each interconnected or interlinked by vectors leading to a converging decision—for medical information. The clinical knowledge graph puts data in context via linking and semantic metadata and provides a framework for data integration, unification, analytics sharing, and decision making in a graphical form. The clinical knowledge graph may be represented in Resource Description Framework (RDF) format which is a standard for data interchange; however, the clinical knowledge graph need not be so limited. The data itself is represented within the clinical knowledge graph as ontologies that are formal descriptions of knowledge as a set of concepts within a domain, in this case medical domain, and the relationships that hold between them is indicated as vectors. To enable such a description, the information is formally specified as components such as individual objects (instances of objects), classes, attributes and relations, as well as restrictions, rules and axioms. As a result, these ontologies provide a sharable and reusable knowledge representation that can also add new knowledge about the data domain.

The clinical knowledge graph has a number of characteristics, including, for example:
capability for data to be explored via structured queries;
graph capability, allowing data to be analyzed as any other network data structure; and
knowledge base, containing formal semantics, which can be used to interpret the data and infer new facts.

The clinical knowledge graph (111) and clinical knowledge graph (106A) represent a collection of available clinical knowledge or data in an interlinked descriptive form comprising symptoms, causation, diseases, related observations, treatment available and other clinical information regarding. The clinical knowledge graph (106A) enable the links (that can be considered vectors) to be traversed based on queries and responses to converge on a diagnosis of a disease for a patient. An example of the clinical knowledge data and vectors of the clinical knowledge graph 106A are shown in FIG. 6 and discussed below. The clinical knowledge graph 106A enables the medical bot to generate queries and receive responses to traverse the graph to arrive at a probable diagnosis for a patient user and output the diagnosis, a triage for the diagnosis and a health score for the user.

As discussed above, in the present application, general medical knowledge usable for initial diagnosis of a problem is input, by downloading the relevant section (106A) of the full clinical knowledge graph (111) from the cloud based on the initial natural language input of the patient user as a searchable clinical knowledge graph (106A). Using queries developed based on interrelationships between the interconnected data elements of the knowledge graph (106A) and responses received for the queries the knowledge graph is traversed. This, as indicated above, leads via a series of linkages to a probable final diagnosis and further to an estimation of disease risks and health score of a user.

A typical example of traversing a simple knowledge graph 106A (only a two level knowledge graph is shown in the example, for simplicity, though a typical knowledge graph may have a large number of linked levels, linked by relational vectors that identify a relationship between the levels of the graph). In the example in FIG. 6, using user input into the input level that is a symptom level provides multiple vector linkages to possible diagnosis using the input symptoms. In a typical case a large number of diagnosis possibilities will exist. For simplicity only four diagnostic possibilities are shown and the response to queries shown in FIG. 4 are used in FIG. 6.

In the example shown in FIG. 6, the user provides an initial input of bloating (606) and stomach pain (604). Initially, the possible linked diagnosis set, at a top level within the knowledge graph 106A, are identified using the symptom inputs provided by the user. In this example, the diagnostic set include any of typhoid (611), food poisoning (612), gastritis (613), and irritable-bowel syndrome (614). By further eliciting the information that vomiting (603) is not a current symptom by the patient in response A1 (408) to query Q1 (401) does not help eliminate any of the possible four diagnoses, but the answer A2 (409) that there is no fever to query Q2 (402), enables typhoid (611) to be eliminated as a diagnosis as the vector linkage from the symptom fever (601) to diagnosis typhoid (611) is negated. The third query Q3 (403) is for clarifying the initial input stomach pain since typhoid (611) has been eliminated as a possible diagnosis, and provides the answer A3 (410) that the ‘pain is intermittent,’ which eliminates food poisoning (612) as a diagnosis, leaving only gastritis (613) and irritable bowel syndrome (614) as possibilities. Query Q4 (404) and its response A4 (411) in the negative helps eliminate gastritis (613) as a possible diagnosis by negating the vector link from symptom loose motion (602) to the gastritis diagnosis (613). The only remaining diagnosis having vector linkage to existing symptoms is irritable bowel syndrome (614). The next three queries Q5 (405), Q6 (406) and Q7 (407) and answers A5 (412), A6 (413) and A7 (414) to the user are used to re-enforce the diagnosis arrived at by specific questions that lead possible additional symptoms that denote the diagnosis to confirm the diagnosis arrive at as the most probable diagnosis—that of irritable bowel syndrome (614).

The most probable diagnosis arrived at from the questions generated and answers received is now fed to the clinical knowledge graph (106A) to search out a treatment and recommend a medication for the diagnosed ailment and provide the information to the patient.

In one embodiment, the policy module 105 is able to validate or eliminate the linkages. This is done by eliminating one by one the linkage between the symptoms and the set of diagnosis possibilities identified. The eliminated possibilities are shown as dashed lines and the positive links are shown as solid lines. The process is continued till only one possible diagnosis is left. The possible diagnosis left is considered the possible final diagnosis or the most probable diagnosis. In this case the ‘irritable bowel syndrome’ (614) is shown as the final possible diagnosis. The final possible diagnosis is fed to the diagnosis module (108) which goes back to the knowledge graph (106A) to extract a health score, a risk for future disease and a triage including medication for the diagnosed disease for the user patient. If the knowledge graph is unable to reach a convergence, the triage will request for further investigations and consultation and arrange an appointment with a doctor at an available time. Also based on the possible final diagnosis the triage will typically provide an individual treatment plan, a doctor visit or a hospital admission.

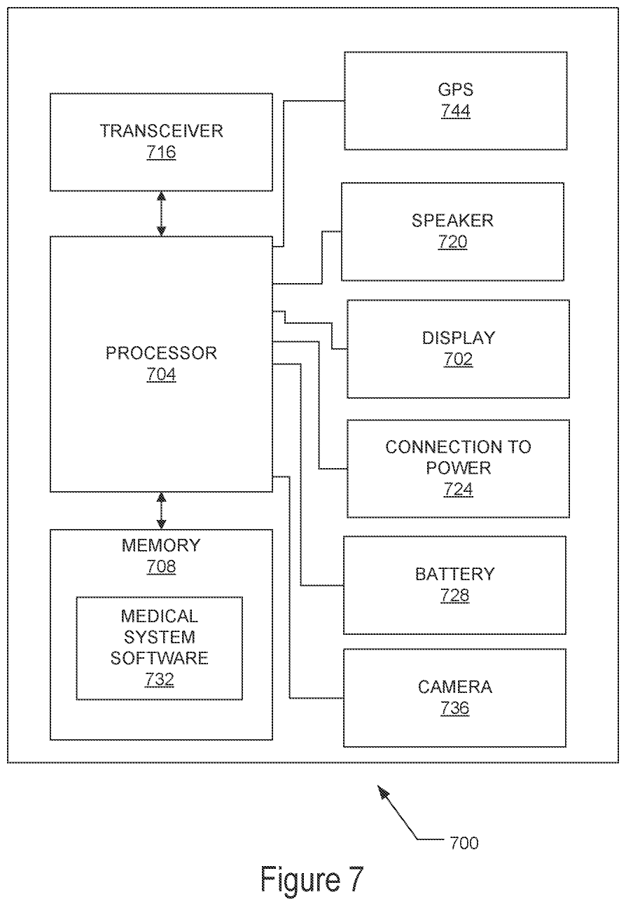

FIG. 7 schematically illustrates a personal communication device including the medical bot or intelligent medical system in accordance with embodiments of the invention. The device 800 may be configured such that the user can download the medical bot to their personal communication device (e.g., smart phone, tablet, etc.) or the device 700 may be a standalone device used only for multiple users (e.g., a tablet used as kiosk).

As shown in FIG. 7, the device 700 includes a display 702, a processor 704, a memory 708, one or more communications transceiver 716, and a speaker 720. As shown, for example, in FIG. 7, the transceiver 716 is coupled to the processor 704. The memory 708, speaker 720 and display 702 are also coupled to the processor 704. The display 704 is also a user interface. The device 700 may further include a connection for an external power supply 724 and an internal battery 728. It will be appreciated that the device 700 may include additional components than those illustrated in FIG. 7.

The display 702 may be a touchscreen that enables the user to use a virtual keyboard to type words, numbers, and other characters to input natural language queries and responses, as discussed herein.

The transceiver 716 may be a dual-band transceiver (or may include multiple transceivers to allow for dual-band or multi-band communications). In some embodiments, the device 700 includes the transceiver 716 is a cellular transceiver to communicate over a cellular network. Additionally or alternatively, the device 700 may include a Wi-Fi or other wireless transceiver and/or an Ethernet connector or other wired connectivity to provide communication connectivity to the device 700.

The processor 704 is configured to operate using the intelligent medical system or medical bot software 732 stored in memory 708 and executed by the processor 704 so that the device 700 can be used to provide the diagnosis and treatment plan as discussed elsewhere herein.

As shown in FIG. 7, the device 700 may also include a camera 736 coupled to the processor 704 to allow for video or image capture near the device 700.

The device 700 may also include a GPS device 744 (or other geo-location sensor) coupled to the processor 704 to collect information about the current location of the device 700. The software 732 executed by the processor 704 can use the geo-location information to identify medical language for a particular location or region where the user is located.

The memory 708 may include computer-readable medium on which is stored one or more sets of instructions (e.g., software 732) embodying any one or more of the methodologies or functions described herein. The software 732 may reside, completely or at least partially, within the memory 808 and/or within the processor 704 during execution thereof by device 700, the memory 708 and the processor 704 also constituting computer-readable media. The software 732 may further be transmitted or received over via the transceiver 716.

One or more of the methodologies or functions described herein may be embodied in a computer-readable medium on which is stored one or more sets of instructions (e.g., software). The software may reside, completely or at least partially, within memory and/or within a processor during execution thereof. The software may further be transmitted or received over a network.

It should be understood that components described herein include computer hardware and/or executable software code which is stored on a computer-readable medium for execution on appropriate computing hardware.

The terms “computer-readable medium” should be taken to include a single medium or multiple media that store the one or more sets of instructions. The term “computer-readable medium” shall also be taken to include any non-transitory storage medium that is capable of storing, encoding or carrying a set of instructions for execution by a machine and that cause a machine to perform any one or more of the methodologies described herein. The terms “computer-readable medium” or “machine readable medium” shall accordingly be taken to include, but not be limited to, solid-state memories, and optical and magnetic media. For example, “computer-readable medium” or “machine readable medium” may include Compact Disc Read-Only Memory (CD-ROMs), Read-Only Memory (ROMs), Random Access Memory (RAM), and/or Erasable Programmable Read-Only Memory (EPROM). In other embodiments, some of these operations might be performed by specific hardware components that contain hardwired logic. Those operations might alternatively be performed by any combination of programmable computer components and fixed hardware circuit components.

Although a number of possible implementations have been mentioned, these are presented merely for the sake of explanation and teaching, and are not limitative. Moreover, an implementation of an apparatus that falls within the inventive concept does not necessarily achieve any of the possible benefits outlined above: such benefits are dependent on the specific use case and specific implementation, and the possible benefits mentioned above are simply examples.

Although the concepts have been described above with respect to the various embodiments, it is noted that there can be a variety of permutations and modifications of the described features by those who are familiar with this field, only some of which have been presented above, without departing from the technical ideas and scope of the features, which is defined by the appended claims.

Further, while this specification contains many features, the features should not be construed as limitations on the scope of the disclosure or the appended claims. Certain features described in the context of separate embodiments can also be implemented in combination. Conversely, various features described in the context of a single embodiment can also be implemented in multiple embodiments separately or in any suitable sub-combination. Accordingly, other implementations are within the scope of the claims that follow.

What is claimed is:

1. A system comprising:
a personal medical-bot instantiated on a communication enabled device having processing capability, the medical bot system comprising:
a module for accepting natural language inputs relating to medical issues from patients; and
a module for parsing the natural language inputs to extract relevant medical information;
a clinical knowledge graph comprising medical vector information relating to the natural language inputs.

2. The system of claim 1, wherein the personal medical-bot system further comprises:
a module for querying the patient and receiving responses to the queries using the clinical knowledge graph; and
a module for generating a diagnosis for the patient and a health score for the patient using the medical information and the responses using the clinical knowledge graph.

3. The system of claim 1, wherein the relevant medical information comprises information on the current illness of the patient, medically relevant personal physical features of the patient, and medically relevant behavioral characteristics of the patient.

4. A method comprising:
generating a medical history of a patient using medical-bot artificial intelligence (AI) and a clinical knowledge graph.

5. The method of claim 4, wherein the method of generating the medical history of the patient comprises:
using the AI and the clinical knowledge graph to generate queries for the patient based on prior input from the patient;
receiving responses to the queries from the patient;
combining the responses from the patient with medical vector information contained in the clinical knowledge graph to generate a health score, a diagnosis and a treatment plan for the patient.

6. The method of claim 5, wherein the diagnosis and the treatment plan is for immediate implementation.

7. The method of claim 5, wherein the health score provides a measure of risk the patient has for contracting a future illness.

8. A method comprising:
generating a medical history of a patient using medical-bot artificial intelligence (AI) and a clinical knowledge graph by:
parsing, using the medical bot and the AI, natural language inputs from the patient;
generating additional queries for the patient based on the parsed natural language inputs from the patient;
receiving responses from the patient in response to the queries;
combining the patient responses with medical vector information contained in a clinical knowledge graph to generate a patient risk index that estimates a probability of a future disease for the patient and an overall health score for the patient.

9. A system comprising:
a medical bot comprising artificial intelligence (AI) enabled to receive and parse natural language inputs from an individual respondent; and
a clinical knowledge graph containing medical information with vector linkages;
wherein the medical bot enabled to generate queries to the individual respondent and extract medically relevant information on illnesses, physical characteristics and personal behavioral patterns of the individual respondent based on responses to the additional queries;
wherein the medical bot is enabled to use the AI to generate a medical history from the responses and historic data of the individual respondent using the clinical knowledge graph; and
wherein the system is further enabled to combine the medical history of the individual respondent with the medical vector information to generate a patient risk index that estimates a future disease probability for the individual and an overall health score for the individual respondent.
